(19) United States
(12) Patent Application Publication
(10) Pub. No.: US 2007/0060855 A1
(43) Pub. Date: Mar. 15, 2007

(54) Pressure-sensitive adhesive compositions and self-adhering wound dressings comprising same

(75) Inventors: Pak-Tong Leung, Rolling Meadows, IL (US); Michael G. Taylor, Mundelein, IL (US); Phil Shelley, Highwood, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: HOLLISTER INCORPORATED, Libertyville, IL (US)

(21) Appl. No.: 11/494,047

(22) Filed: Jul. 27, 2006

Related U.S. Application Data
(60) Provisional application No. 60/703,407, filed on Jul. 28, 2005.

(51) Int. Cl. A61F 15/00; A61F 13/00

(52) U.S. Cl. 602/41

(57) ABSTRACT

The disclosure generally relates to pressure-sensitive adhesive compositions and self-adhering wound dressings, and more particularly, to pressure-sensitive adhesive compositions comprising a triblock copolymer and a superabsorbent material and self-adhering wound dressings comprising same. The adhesive compositions are skinfriendly and therefore may be used in medical environments where a dressing, bandage, ostomy pouch, continence care appliance, transdermal drug delivery patch, or similar article is intended to be adhered to the skin. The self-adhering wound dressings are also skinfriendly.

### CROSS-REFERENCE TO RELATED APPLICATION

The benefit under 35 U.S.C. §119(e) of U.S. provisional patent application ser. no. 60/703,407 filed Jul. 28, 2005, the entire disclosure of which is incorporated herein by reference, is hereby claimed.

### FIELD OF THE DISCLOSURE

The disclosure generally relates to pressure-sensitive adhesive compositions and self-adhering wound dressings, and more particularly, to pressure-sensitive adhesive compositions comprising a triblock copolymer and a superabsorbent material, and self-adhering wound dressings comprising same.

### BACKGROUND OF THE DISCLOSURE

Pressure-sensitive adhesive compositions are widely used in the medical field to adhesively secure various articles to an individual's skin. Typical pressure-sensitive adhesive compositions, however, often lack ‘skinfriendliness.’ For example, individuals often damage their skin and/or experience substantial discomfort (caused by, for example, the pulling out of hairs) upon removing the article adhesively secured to their skin. The skinfriendliness problem is particularly acute for individuals having conditions involving frequently, repeated removal and replacement of such articles because it may result in skin stripping. Skin stripping occurs when a continual damaging force is applied to the outermost layer of the epidermis, thereby causing a loss of epidermal protective and regulatory functions. Furthermore, the skinfriendliness problem is often exacerbated when the pressure-sensitive adhesive compositions are formulated for extended use.

Pressure-sensitive adhesive compositions containing naturally-derived hydrocolloids are considered desirable for adhesive applications requiring skin contact because they readily absorb moisture (e.g., perspiration). However, pressure-sensitive sensitive adhesive compositions containing naturally-derived hydrocolloids are relatively stiff (i.e., they are not very flexible or conformable) because high loading of the naturally-derived hydrocolloids is necessary to provide the adhesive compositions with sufficient absorption rates. Thus, continued adhesion to moving body parts (such as elbows and knees) can be problematic. Further, naturally-derived hydrocolloids often comprise polysaccharides, modified polysaccharides, and/or other natural products or modified natural products that can leach onto an individual's skin and support microbial growth, e.g., on the individual's skin and/or on the article to be adhered to the individual's skin, and therefore present an additional consideration that needs to be accounted for by the attending health professionals.

Moreover, pressure-sensitive adhesive compositions containing naturally-derived hydrocolloids lack skinfriendliness because they adhere to hairy skin and often cause skin damage and/or substantial discomfort, as previously discussed.

Furthermore, the performance of such pressure-sensitive adhesive compositions containing naturally-derived hydrocolloids can vary widely due to inherent variations because such hydrocolloids are natural products or modified natural products derived from plants, animals, and microbes. Additionally, pressure-sensitive adhesive compositions containing naturally-derived hydrocolloids are typically colored, and thus individuals (and/or their medical providers) cannot visually inspect the skin areas covered or coated with the adhesive composition without removing the article adhesively secured to their skin. Unnecessary removal and replacement of articles adhesively secured to the skin is to be avoided, particularly in view of the skinfriendliness issue.

In various ostomy surgical procedures such as colostomies, ileostomies, and urostomies, a hole is made in the abdomen and a portion of the small intestine, the large intestine, or the ureter is attached to the surface of the skin to create an opening for discharging body wastes. The point at which the ureter or intestine protrudes from the abdominal wall is called a stoma. Pressure-sensitive adhesive compositions have been widely used to adhere waste collection appliances to the peristomal surfaces of individuals who have undergone such surgical procedures. The skinfriendliness problem discussed above can be especially problematic when stoma fluids (which may contain enzymes and/or acidic, gastric juices) contact skin that has already been continuously irritated because of frequent removal and replacement of the waste collection appliances. Injured peristomal skin is a source of irritation and discomfort, and provides an environment for bacteria growth. Thus, the health of the peristomal skin is a primary concern for osotmates and health professionals.

Wound dressings can be provided in many different forms. Conventional wound dressings sold under the BAND-AID® trade name (Johnson & Johnson, NJ) have a plastic film backing, an adhesive layer disposed on the film's inner surface (to adhere the dressing to an individual's skin), and a non-woven pad material attached to the film's inner surface (to absorb exudates from the wound, perspiration, and/or other fluids). When the dressing is applied to the skin of an individual, the nonwoven pad is placed over a wound and in contact with the skin. The porous plastic film backing is designed to allow moisture that is trapped under the dressing to evaporate through its pores. However, moisture trapped under the dressing does not readily evaporate out through the pores, and the absorptive element does not adequately remove fluids from contacting the individual's skin. For example, the skin covered by the wound dressing is often moist and wrinkled, upon visual inspection, after the dressing is removed. A wound may be increasingly subjected to bacteria and a long healing time when the covered skin remains moist.

Another commercially available wound dressing is sold under the RESTORE CX™ trade name (Hollister Incorporated, IL). Such wound dressings generally comprise an adhesive composition comprising a naturally-derived hydrocolloid carried by a film backing. As previously discussed, the naturally-derived hydrocolloids can vary widely due to inherent variations. Additionally, such hydrocolloids can promote microbial growth, as previously discussed. Furthermore, wound dressings comprising such hydrocolloids are generally not capable of absorbing large amounts of exudates, perspiration, and/or other fluids, particularly when constructed as a thin wound dressing which is comfortable for the individual to wear. When such wound dressings are constructed to be thicker (so as to absorb more exudates, perspiration, and/or other fluids), individuals often experience substantial discomfort.

### SUMMARY OF THE DISCLOSURE

The disclosure provides pressure-sensitive adhesive compositions comprising a triblock copolymer and a superabsorbent material. The adhesive compositions are skinfriendly, possess good adhesive strength and good cohesive strength, and have gel-like properties which make them particularly adapted for use in medical environments where a dressing, bandage, ostomy pouch, continence care appliance, transdermal drug delivery patch, or similar article is intended to be adhered to skin. Moreover, the adhesive compositions are able to withstand and/or absorb external moisture so an individual can participate in physical activities where they perspire and/or even shower, without having to remove and subsequently replace the article. Furthermore, the adhesive compositions are translucent, and therefore the skin areas that are covered or coated with the adhesive compositions or wound dressings comprising same can be readily visually inspected (i.e., without removal of the article adhesively secured to the skin). Additionally, the adhesive compositions can help to moisturize the skin, e.g., if formulated, to include an emollient. Also, the properties of the adhesive compositions are more consistent because the adhesive compositions generally do not include large quantities of hydrocolloids that are natural products or modified natural products. Further, the adhesive compositions can be formulated to be substantially free of hydrocolloids that are natural products or modified natural products, and therefore to not support microbial growths.

The disclosure also provides a self-adhering wound dressing comprising a pressure-sensitive adhesive composition as described herein. Such wound dressings are advantageous in that they are able to absorb substantial amounts of exudates, perspiration, and/or other fluids while being comfortable for the individual to wear.

One embodiment according to the disclosure provides a pressure-sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

A further embodiment according to the disclosure provides a waste collection appliance comprising a mounting faceplate and a waste collection pouch comprising a polymeric material, wherein the mounting faceplate includes a layer of pressure-sensitive sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

An additional embodiment according to the disclosure provides a pressure-sensitive sensitive adhesive composition comprising about 0.25 weight percent (wt. %) to about 15 wt. % of a high molecular weight rubber triblock copolymer and about 35 wt. % to about 65 wt. % of a superabsorbent material, wherein the adhesive composition has a compressive force of less than about 15 pounds per square inch (psi) at 10 percent (%) strain.

Yet another embodiment of the disclosure provides a self-adhering wound dressing comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

### DETAILED DESCRIPTION OF THE DISCLOSURE

The disclosure provides pressure-sensitive adhesive compositions comprising a triblock copolymer and a superabsorbent material. The adhesive compositions are skinfriendly, possess good adhesive strength and good cohesive strength, and have gel-like properties which make them particularly adapted for use in medical environments where a dressing, bandage, ostomy pouch, continence care appliance, transdermal drug delivery patch, or similar article is intended to be adhered to skin. Moreover, the adhesive compositions are able to withstand and/or absorb external moisture so an individual can participate in physical activities where they perspire and/or even shower, without having to remove and subsequently replace the article. Furthermnore, the adhesive compositions are translucent, and therefore the skin areas that are covered or coated with the adhesive compositions or wound dressings comprising same can be readily visually inspected (i.e., without removal of the article adhesively secured to the skin). Additionally, the adhesive compositions can help to moisturize the skin, e.g., if formulated to include an emollient. Also, the properties of the adhesive compositions are more consistent because the adhesive compositions generally do not include large quantities of hydrocolloids that are natural products or modified natural products. Further, the adhesive compositions can be formulated to be substantially free of hydrocolloids that are natural products or modified natural products, and therefore to not support microbial growth. Such adhesive compositions are generally considered to be bacteriostatic.

The disclosure also provides self-adhering wound dressings comprising pressure-sensitive adhesives, as described herein.

As used herein, the term “pressure-sensitive adhesive” refers to a material which (1) adheres instantaneously to most substrates with the application of slight pressure and (2) remains permanently tacky.

As used herein, the term “gel-like property” refers to an adhesive material that is soft to the touch and flows easily under pressure. Adhesive compositions having gel-like properties are more flexible and more comfortable during wear than conventional pressure-sensitive adhesive compositions containing naturally-derived hydrocolloids. For example, adhesive compositions having gel-like properties adhere well to hairy skin but are skinfriendly, i.e., such adhesive compositions do not cause substantial skin damage and/or substantial discomfort upon removal. Accordingly, such adhesive compositions are particularly useful for adhering articles that are designed for short term wear (e.g., articles involving multiple daily changes). Additionally, such adhesive compositions do not irritate peristomal skin upon removal and thus are well-suited for ostomy appliance applications. Furthermore, adhesive compositions having gel-like properties do not require heating to adhere to skin and leave a minimum amount of residue upon removal. In various embodiments, the term gel-like property refers to an adhesive material having a compressive force of less than about 15 pounds per square inch (psi), less than about 10.0 psi, less than about 7.5 psi, and/or less than about 5 psi at 10 percent (%) strain.

As used herein, the term “high molecular weight rubber” refers to materials having a viscosity greater than 1000 centipoises (cps) in toluene at a concentration of 20% by weight (at 25° C.).

As used herein, the term “superabsorbent material” refers to materials capable of absorbing and retaining large volumes of water (or aqueous solutions). In various embodiments, the term superabsorbent material refers to a material capable of absorbing and retaining distilled water in an amount at least about 200 times, at least about 300 times, and/or at least about 400 times its weight. In other embodiments, the term superabsorbent material refers to, a material capable of absorbing and retaining 1 wt. % sodium chloride solution in an amount at least about 10 times, at least about 20 times, and/or at least about 30 times its weight.

In the context of this disclosure, “substantially free of hydrocolloids that are natural products or modified natural products” means that the adhesive compositions (or the self-adhering wound dressings comprising same) contain less than about 2 wt. % of such hydrocolloids, based on the total weight of the adhesive composition. More preferably, the adhesive compositions contain less than about 1 wt. %, and most preferably less than about 0.25 wt. % of such hydrocolloids. Hydrocolloids that are natural products or modified natural products generally comprise polysaccharides, polysaccharide derivatives, or proteins (e.g., gelatin).

The adhesive compositions described herein are useful to adhere waste collection appliances, dressings, prosthetic and other metal and/or plastic devices to the skin. For example, the adhesive compositions can be used to adhere ostomy appliances, such as ileostomy, colostomy and urostomy appliances to the skin; to attach monitoring devices for patient monitoring, e.g., heartbeat monitoring and brain wave monitoring to the skin; to affix surgical dressings and pads to the skin; to adhere external prosthetic devices; to adhere hairpieces to the skin (e.g., the scalp); and to adhere medical tape to the skin. In ostomy care, the adhesive compositions are useful for adhering one piece closed pouches for short term wear (i.e., pouches that are intended to be changed multiple times each day) including both flat and convex products; drainable one-piece products with either flat or convex barriers for short or long term wear; two piece ostomy care products; pediatric ostomy systems, including one-piece and two piece pouches; stoma cap pouches; skin barrier sheets; skin barrier rings; and paste and skin gel products. In continence care, the adhesive compositions are useful for adhering male external catheters; silicone male external catheters; female continence devices; fecal pouches; retracted penis pouches; and glans caps. In wound care, the adhesive compositions are useful for adhering tube attachment devices such as nasogastric tube attachment devices, endotracheal tube attachment devices, and other similar attachment devices; wound care dressings; scar therapy dressings; island dressings; reclosable wound covers; transparent polyurethane thin film wound dressings; bandages, and other wound dressings, e.g., burn dressings; surgical tape, and underlayment tape for athletes hands and ankles. The adhesive compositions are also useful for adhering transdermal drug delivery patch systems, e.g., to administer nitroglycerin or other drugs such as morphine, Dramamine, contraceptive drugs, or nicotine patch medicaments. Further, the adhesive compositions can be used to adhere foam dressings to secure I.V. needles and catheters to the body. Additionally, the adhesive compositions can be used to adhere a nipple shield to a breast or to secure a feeding tube thereto for breast feeding.

The self-adhering wound dressings comprising a pressure-sensitive adhesive composition, as described herein, can be used in generally known wound care applications. Accordingly, the wound dressings can be used to treat a variety of conditions including but not limited to those associated with scarring, bums, abrasions, lacerations, incisions, skin grafts, dermal ulcers, rashes, animal bites, and insect bites. In one aspect of this embodiment, the self-adhering wound dressing consists essentially of a pressure-sensitive adhesive composition described herein (and does not include any other component as an absorptive element). It is generally understood that the self-adhering wound dressing consisting essentially of a pressure-sensitive adhesive composition described herein may further include a film backing (oriented away for the wound). In contrast, adhesive compositions disclosed herein are typically used to adhere a separate article to an individual's skin (i.e., to bear a separate load).

The self-adhering wound dressings can be advantageously constructed to be comfortable for the individual to wear. In one aspect, the self-adhering wound dressings have a thickness of between about 5 mils and about 50 mils; in another aspect the wound dressing have a thickness of greater than 50 mils. The thickness of the adhesive layer of the wound dressings can be varied to provide different levels of fluid absorption. For example, dressings having an adhesive layer thickness between about 5 mils and 10 mils may be most suitable for wounds with little or no fluid exudates. Dressings having an adhesive thickness between about 10 mils and about 30 mils are suitable for wounds with moderate exudates levels. Finally, dressings having an adhesive thickness greater than about 30 mils, greater than about 40 mils, and/or greater than about 50 mils are generally preferred for wounds with moderate to heavy exudates levels.

Advantageously, the wound dressings can be formulated to be substantially free of hydrocolloids that are natural products or modified natural products, and therefore not to support microbial growth. Because the adhesive compositions in accordance with the disclosure can be formulated to have substantial absorptive capacity, the self-adhering wound dressings can be formulated to absorb light fluid exudates or heavy fluid exudates (depending on the specific wound and the amount of superabsorbent material in the composition).

The high molecular weight rubber triblock copolymers typically have an A-B-A block polymer configuration. The A blocks are non-elastomeric polymers having glass transition temperatures above 20° C. (as homopolymers). The non-elastomeric polymers may comprise homopolymers or copolymers of vinyl monomers including but not limited to vinyl arenes, vinyl pyridines, vinyl halides, vinyl carboxylates, and the like, as well as homopolymers or copolymers of acrylic monomers including but not limited to acrylonitrile, methacrylonitrile, acrylic acid esters, and the like. Suitable vinyl arenes include but are not limited to styrene, vinyl toluene, vinyl xylene, ethyl vinyl benzene, vinyl naphthalene, and the like. Other non-elastomeric polymers may be derived from monomers including but not limited to alpha olefins, alkylene oxides, acetals, urethanes, and the like. Styrene is a preferred A block.

The B blocks are elastomeric polymers having glass transition temperatures between about −30° C. and 10° C., or from about −20° C. and about 0° C. Suitable B blocks include but are not limited to polymers derived from monomers including but not limited to isoprene and butadiene. In one embodiment, the B blocks comprise both isoprene and butadiene units. The B block polymers may be partially or substantially hydrogenated as taught, for example, in U.S. Pat. No.3,700,633. Thus, the pressure-sensitive adhesive composition may contain partially or substantially hydrogenated A-B-A copolymers or mixtures thereof.

When the hydrogenation reaction is conducted, selected reaction conditions are typically employed to hydrogenate the elastomeric B blocks without modifying the non-elastomeric A blocks. However, the hydrogenation reaction conditions may be chosen to hydrogenate both the elastomeric and non-elastomeric blocks of the triblock copolymer to (practically) the same extent, which may be either partially or substantially complete. Hydrogenated copolymers are preferred to minimize degradation during processing.

The triblock copolymers typically have a ratio of non-elastomeric end blocks (A blocks) to elastomeric center blocks (B blocks) between about five to about 95 (or less) and about 40 to about 60 (or higher). The triblock copolymers typically comprise from about 0.25 wt. % to about 15 wt. % of the pressure-sensitive adhesive. In other embodiments, the triblock copolymers comprise from about 1 wt. % to about 10 wt. %, and/or from 2 wt. % to about 8 wt. % of the pressure-sensitive adhesive.

Examples of suitable triblock copolymers include substantially hydrogenated random midblock copolymers such as polystyrene-b-poly(ethylene/propylene)-b-polystyrene block copolymers (“substantially hydrogenated SEPS”), polystyrene-b-poly (ethylene/butylene)-b-polystyrene block copolymers (“substantially hydrogenated SEBS”), polystyrene-b-poly(ethylene-ethylene/propylene)-b-polystyrene block copolymers (“substantially hydrogenated SEEPS”) such as those sold under the SEPTON® trade name (SEPTON Company of America, TX; Kuraray Co. Ltd., Elastomer Company, Japan). Specific exemplary SEPTON® polymers include SEPTON® 2002, SEPTON® 2004, SEPTON® 2005, SEPTON® 2006, SEPTON® 2007, SEPTON® 2063, SEPTON® 2014, SEPTON® 8004, SEPTON® 8006, SEPTON® 8007, SEPTON® 8104, SEPTON® 4033, SEPTON® 4044, SEPTON® 4055, SEPTON® 4077, and SEPTON® 4099. Other suitable triblock copolymers include styrenic block copolymers such as the styrene-butadiene-styrene block copolymers (“SBS”), styrene-isoprene-styrene block copolymers (“SIS”), and styrene-ethylene/butylenes-styrene block copolymers (“SEBS”) sold under the KRATON® trade name (KRATON Polymers U.S. LLC, Houston, Tex.). Specific exemplary KRATON® polymers include KRATON® D1101, KRATON® D1102, KRATON® D1107P, KRATON® D1111, KRATON® D1112P, KRATON® D1113P, KRATON® D1117P, KRATON® D1119P, KRATON® D1133, KRATON® D1193P, KRATON® D4141, KRATON® D4150, KRATON® D4433, KRATON® G1650, KRATON® G1651, KRATON® G1652, KRATON® G1654, KRATON® G1657, KRATON® G1726, KRATON® FG1901, and KRATON° FG1924X. Other triblock copolymers can also be utilized provided that such copolymers have a viscosity greater than 1000 cps in toluene at a concentration of 20% by weight (at 25° C.). The substantially hydrogenated SEEPS polymers are preferred because of their high tensile strength and high capacity for oil absorption, which gives the adhesive compositions a soft to the touch feel.

The superabsorbent material typically comprises a conventionally known polymeric superabsorbent material. Exemplary polymeric superabsorbent materials include but are not limited to sodium polyacrylates, acrylic acid-vinyl alcohol copolymers, cross-linked polyvinyl alcohols, polyacrylic acids, polyamides, polyvinyl pyrrolidones, and starch-acrylic acid graft copolymers. Suitable polymeric superabsorbent materials include those sold under the AQUA KEEP® trade name (Sumitomo Seika Chemicals Co., Ltd., Japan; KOBO Products, Inc., NJ; Absorbent Technologies, Inc., IA) such as AQUA KEEP® 10 SH-NF. The particle size of the superabsorbent material can be less than about 900 microns (μ), less than about 240μ, less than about 140μ, and/or less than about 75μ. Superabsorbent materials having a particle size of less than about 75μare generally preferred.

If the amount of superabsorbent material is greater than about 65 wt. %, the adhesive composition may lose its advantageous gel-like properties. On the other hand, the adhesive composition does not have a sufficient absorption capacity when the amount of superabsorbent material is less than about 35 wt. %. Thus, the adhesive compositions comprise from about 35 wt. % to about 65 wt. %, 35 wt. % to about 60 wt. %, about 40 wt. % to about 55 wt. %, about 42.5 wt. % to about 52.5 wt. %, and/or about 45 wt. % to about 50 wt. % of the superabsorbent material. In one embodiment, the superabsorbent material is substantially dried in an oven in order to remove any residual water prior to being added to the adhesive composition.

The high molecular weight rubber diblock polymers typically have an A-B block polymer configuration where A and B are as previously described. Preferred A-B block copolymers include those sold under the KRATON® trade name (KRATON Polymers U.S. LLC, Houston, TX). Specific exemplary polymers include KRATON® G1701 and KRATON® G1702, which are both styrene ethylene propylene (“SEP”) diblock polymers. Another suitable exemplary polymer is KRATON® D 1118, which is a styrene butadiene diblock polymer. The adhesive composition typically contains from 0 wt. % to about 15 wt. %, about 0.50 wt. % to about 12 wt. %, and/or about 1.0 wt. % to about 10 wt. % of the high molecular weight diblock rubber.

While the adhesive formulation preferably-contains high molecular weight diblock rubber polymer as previously set forth, the diblock polymer may be replaced entirely or partially with another high molecular weight polymer that is compatible with the system. For example, the adhesive composition may contain about 2 wt. % to about 10 wt. % of a high molecular weight polymer including but not limited to polymers such as polyisobutylene, polyisoprene, and styrene/butadiene copolymers.

The end block resin is typically a resin which resides predominantly in the non-elastomer domains of the adhesive composition after cooling. Representative resins include primarily aromatic resins based on mixed C₉ petroleum distillation streams including but not limited to homo or copolymers of vinyl toluene, styrene, alpha-methyl styrene, coumarone, and indene. Preferred end block resins include alpha-methyl styrene based polymers sold under the KRISTALEX® trade name (Eastman Chemical Company, TN). Specific exemplary polymers include KRISTALEX® 3085 and KRISTALEX® 5140. The adhesive composition typically contains from 0 wt. % to about 20 wt. %, about 1.0 wt. % to about 15 wt. %, and/or about 2.0 wt. % to about 12 wt. % of the end block resin.

The liquid diluent is typically primarily hydrocarbon in character, and should be compatible with the polymer midblock of the triblock copolymer. Suitable liquid diluents include plasticizers (such as paraffinic and naphthenic petroleum oils), white petroleum mineral oils, and liquid tackifiers such as synthetic liquid oligomers of polybutene, polypropene, polyterpene, etc. White paraffinic oils are preferred. The adhesive composition typically contains from 0 wt. % to about 40 wt. %, about 2.0 wt. % to about 35 wt. %, and/or about 5.0 wt. % to about 30 wt. % of the liquid diluent.

The solid tackifier is a resin having a Ring and Ball softening point (as determined in accordance with ASTM Standard Test Method D36) above 25° C., which is compatible with the polymer midblock of the triblock copolymer. Suitable solid tackifiers include (1) natural or modified rosins such, for example, as gum rosin, wood rosin, tall-oil rosin, distilled rosin, hydrogenated rosin, dimerized rosin, and polymerized rosin; (2) glycerol and pentaerythritol esters of natural or modified rosins, such, for example as the glycerol ester of pale rosin, the glycerol ester of wood rosin, the glycerol ester of hydrogenated rosin, the glycerol ester of polymerized rosin, the pentaerythritol ester of hydrogenated rosin, and the phenolic-modified pentaerythritol ester of rosin; (3) copolymers and terpolymers of natural terpenes, e.g., styrene/terpene and alpha methyl styrene/terpene; (4) polyterpene resins having a softening point (as determined in accordance with ASTM Standard Test Method E28-99) of from about 80° C. to 150° C.; (5) phenolic modified terpene resins and hydrogenated derivatives thereof; (6) aliphatic petroleum hydrocarbon resins and hydrogenated aliphatic petroleum hydrocarbon resins having a Ring and Ball softening point (as determined in accordance with ASTM Standard Test Method D36) between about 70 ° C. and 135 ° C.; (7) alicyclic petroleum hydrocarbon resins and the hydrogenated derivatives thereof; and (8) aliphatic/ aromatic or cycloaliphatict aromatic copolymers and their hydrogenated derivatives.

Preferred tackifiers include polyterpene resins such as those sold under the PINOVA™ PICCOLYTE® trade name (Hercules Inc., DE), for example, PICCOLYTE® A125; aliphatic resins such as those sold under the WINGTACK® trade name (Goodyear Chemical, OH), for example, WINGTACK® 95; cycloaliphatic resins such as those sold under the EASTOTAC® trade name (Eastman Chemical Company, TN), for example, EASTOTAC® H100; and aliphatic/aromatic or cycloaliphatic/aromatic resins such as those sold under the ESCOREZ® trade name (ExxonMobil Chemical Company, TX), for example ESCOREZ® 5340, ESCOREZ® 5400, and ESCOREZ® 5415. The desirability and selection of the particular tackifying agent is largely dependent upon the specific block copolymers employed. The adhesive composition typically contains from 0 wt. % to about 35 wt. %, about 2.0 wt. % to about 30 wt. %, and/or about 5.0 wt. % to about 25 wt. % of the tackifier.

The natural product or modified natural product containing hydrocolloid should form a gel in water. Suitable hydrocolloids include naturally derived products such as pectin, gelatin, starches, guar gum, gum arabic, locust bean gum, gum karaya, alginic acid and its sodium and/or calcium salts. Synthetic hydrocolloids such as sodium carboxymethyl cellulose, cross-linked or crystalline sodium carboxymethyl cellulose, polyvinyl alcohol, polyvinyl pyrrolidone, high molecular weight polyethylene glycols, and high molecular weight polypropylene glycols may also be used. When present, the hydrocolloid content can also be a blend of such natural and synthetic hydrocolloids. The adhesive composition typically contains from 0 wt. % to about 20 wt. %, about 0 wt. % to about 15 wt. %, about 0 wt. % to about 10 wt. %, and/or about 0 wt. % to about 5 wt. % of the natural product- or modified natural product-containing hydrocolloid. When the adhesive composition contains greater than about 30 wt. % of such hydrocolloids, the translucency of the adhesive composition can be negatively affected. Furthermore, the skinfriendliness of the composition is often negatively affected when greater than about 30 wt. % of such hydrocolloids is included in the adhesive composition.

The semi-solid hydrocarbon enhances sprayability of the adhesive compositions, and is typically petrolatum. The adhesive composition typically contains from 0 wt. % to about 10 wt. %, about 0 wt. % to about 8 wt. %, and/or about 0 wt. % to about 7 wt. % of the semi-solid hydrocarbon.

Convenient sources of the high molecular weight diblock rubber, the end block resin, the diluent, and the solid tackifier are hot melt adhesive compositions sold under the DERMA-TAK® trade name (National Starch & Chemical Company, Bridgewater, N.J.). Specific exemplary products including the high molecular weight diblock rubber, the end block resin, the diluent, and the solid tackifier include DERMA-TAK® 34-541B, DERMA-TAK® 34-542B, and DERMA-TAK® 34-447A. Other hot melt adhesives may also be used in combination with a superabsorbent material to provide pressure-sensitive adhesive compositions in accordance with the disclosure. The pressure-sensitive compositions according to the disclosure generally comprise about 35 wt. % to about 65 wt. % of a hot melt adhesive composition, about 35 wt. % to about 65 wt. % of a superabsorbent material (and/or in accordance with the other ranges provided above), 0 wt. % to about 20 wt. % of a hydrocolloid comprising a natural product or a modified natural product (and/or in accordance with the other ranges provided above), and 0 wt. % to about 10 wt. % of a semi-solid hydrocarbon (and/or in accordance with the other ranges provided above).

The pressure-sensitive adhesive compositions in accordance with the disclosure may also further comprise additives such as anti-oxidants, waxesi and the like.

One embodiment of the disclosure provides a pressure-sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

In a further embodiment, the disclosure provides a waste collection appliance comprising a mounting faceplate and a waste collection pouch comprising a polymeric material, wherein the mounting faceplate includes a layer of pressure-sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

The waste collection pouch may be an ostomy appliance including but not limited to an ileostomy appliance, a colostomy appliance, and a urostomy appliance. The pouch may be permanently connected to the mounting faceplate (“a one-piece appliance”) as described in U.S. Pat. No. 6,830,565, which is incorporated herein by reference in its entirety. Alternatively, the pouch may be removably coupled to the mounting faceplate (“a two-piece appliance”) as described in U.S. Pat. No. 4,419,100, which is incorporated herein by reference in its entirety. The pouch (whether of one-piece or two-piece construction) is attached to the peristomal surfaces of an individual by the mounting faceplate, and is fitted over and around the stoma to collect waste. The disclosed pressure-sensitive adhesive composition is designed to strongly adhere the faceplate to the abdomen while being capable of being easily removed. Thus, the disclosed adhesive composition minimizes the potentially hazardous effects caused by stoma output.

Of course, the disclosed adhesive compositions may also be used to adhere other articles to skin including but not limited to dressings, bandages, ostomy pouches, continence care appliances, transdermal drug delivery patches, as described above. Further, the adhesive compositions described herein can be formulated as self-adhering wound dressings, as previously described.

An additional embodiment according to the disclosure provides a pressure-sensitive sensitive adhesive composition comprising about 0.25 weight percent (wt. %) to about 15 wt. % of a high molecular weight rubber triblock copolymer and about 35 wt. % to about 65 wt. % of a superabsorbent material, wherein the adhesive composition has a compressive force of less than about 15 pounds per square inch (psi) at 10 percent (%) strain.

Yet another embodiment of the disclosure provides a self-adhering wound dressing comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

Pressure-sensitive adhesive compositions and self-adhering wound dressings in accordance with the disclosure can be better understood in light of the following examples. However, the foregoing description and the following examples are merely illustrative, and therefore no unnecessary limitations should be understood therefrom as numerous modifications and variations are expected to occur to those skilled in the art.

### EXAMPLE 1

### Compression Method for Measuring the Resistance Force of an Adhesive Composition

Using a tensile tester, a 90 mil thick disc of adhesive sandwiched between two polyester release liners was put under compression in order to generate a stress (force or modulus) and strain response curve. The strain is defined as the change in thickness (or displacement) over the original thickness and is reported as a percentage (%). Thus, the strain equals the change in disc thickness divided by the original disc thickness. The force or modulus number at 10% strain or 20% strain can be extrapolated from the generated data.

The tensile tester compression speed was 1 inch per minute. The relative humidity was 50%, and the temperature was 72° F.

### EXAMPLE 2

### Exemplary Pressure-sensitive Adhesive Compositions and Wound Dressing

Exemplary pressure-sensitive adhesive composition examples A-E, self-adhering wound dressing example F, and a comparative example were prepared in accordance with table 1, below. The exemplary compositions were tested for various physical properties including adhesion force and fluid absorption capacity. One of the exemplary compositions was also tested for properties demonstrating its skinfriendliness relative to a commercially available adhesive composition containing a hydrocolloid comprising a natural product or a modified natural product.

A model REE6 Prep-Mixer (C. W. Brabender Instruments, Inc., N.J.) was used to blend the components to yield adhesive composition examples A-E in accordance with the invention, wound dressing example F, and a comparative example. Examples A-F were prepared by adding a first portion of the superabsorbent powder (approximately 50 wt. %), a first portion of the hot melt adhesive composition (approximately 50 wt. %), the remaining portion of the hot melt adhesive composition, the petrolatum (if present), and the remaining 50% of the superabsorbent powder to the mixer. Mixing was performed until the mixture was homogeneous. The mixtures were then removed from the mixer and allowed to equilibrate at room temperature before any additional testing was undertaken.

Thin (18 mil) samples of adhesive compositions A-F were laminated to 0.5 mil polyurethane film. The polyur&thane films carrying adhesive compositions were subsequently laminated to a polyethylene nonwoven tape for adhesion to steel testing. For adhesion to human skin testing, the polyurethane films carrying adhesive compositions were laminated to an adhesive tape backed with an ethylene methyl acrylate copolymer film.

Adhesion to Steel

Peel adhesion force from stainless steel was measured using a tensile tester, peeling one inch wide samples at 180 degrees and a separation speed of 12.5 inches per minute.

Fluid Absorption

Fluid absorption testing was performed following the British Pharmacopoeia 1993 monograph. Circular samples of 10 cm² area were exposed to 0.9% sodium chloride solution for 24 hours at 37° C. Fluid absorption was calculated as the weight gain of the sample divided by the area of the sample (10 cm²) to yield values having units of g/cm² .

The data presented in Table 1 demonstrate that the fluid absorption capacity of the pressure-sensitive adhesive compositions and the wound dressing in accordance with the disclosure were increased as the content of superabsorbent material in the mixture is increased. The fluid absorption capacity of the pressure-sensitive adhesive compositions was also increased by adding petrolatum (as shown by comparing Examples A-C). The adhesion peel force against steel was generally decreased by adding petrolatum to the adhesive compositions in accordance with the disclosure. Surprisingly, the adhesion forces of samples A, B, and D-F was greater than the adhesion force of comparative example F.

TABLE I

| | Example Compositions | | | | | | |
|---|---|---|---|---|---|---|---|
| | Example | Example | Example | Example | Example | Example | Comparative |
| | A | B | C | D | E | F | Example |
| Ingredient | Wt. % | Wt. % | Wt. % | Wt. % | Wt. % | Wt. % | Wt. % |
| DERMA-TAK ® 34-542B | 56 | 52.5 | 49 | 54 | 52 | 0 | 100 |
| DERMA-TAK ® 34-447A | 0 | 0 | 0 | 0 | 0 | 40 | 0 |
| Superabsorbent, | 44 | 44 | 44 | 46 | 48 | 60 | 0 |
| AQUA KEEP ® | | | | | | | |
| SAP (10SH-NF) | | | | | | | |
| Petrolatum | 0 | 3.5 | 7 | 0 | 0 | 0 | 0 |
| Fluid absorbed | 0.44 | 0.55 | 0.61 | 0.63 | 0.80 | 1.30 | Not |
| (g/cm²) | | | | | | | measured |
| Adhesion to steel | 5.20 | 4.32 | 2.67 | 5.21 | 5.21 | 5.01 | 3.1 |
| peel force (pounds | | | | | | | |
| per inch) | | | | | | | |

Skin Adhesion Testing Methods

Performance on human skin was measured by applying rectangular 1 inch by 3 inch samples of ethylene methyl acrylate copolymer tape/polyurethane film carrying adhesive compositions (as previously described) to the abdomens of subjects. Example D, described above, was compared to a skin barrier adhesive material containing a hydrocolloid comprising a natural product or a modified natural product, which is commercially available under the SOFTFLEX® trade name (Hollister Incorporated, IL). The samples were adhered to the subjects' skin for variable time periods (30 minutes, 6 hours, 24 hours, 48 hours, and 72 hours) before removal. The samples were removed by peeling with a tensile tester at 90 degrees from the skin using a separation speed of 150 mm per minute. The force required to peel the sample from the skin was recorded in units of grams force at about 0.5 hours, about 6 hours, about 24 hours, about 48 hours, and about 72 hours.

Additionally, Transepidermal Water Loss (TEWL) measurements were made to assess damage to the stratum corneum (or outermost layer of epidermis) caused by removal of the adhesive compositions. The TEWL measurement provides a noninvasive method for determining whether the barrier function of the stratum corneum has been disrupted by the application and/or removal of the adhesive compositions adhered to the subjects' skin. Damage to the stratum corneum is demonstrated by elevated water loss rates (relative to the water loss rates observed prior to injury). TEWL measurements were made prior to application of the adhesive strips- and following removal at the same sites to determine the increase in TEWL rates resulting from removal of the adhesives.

Additionally, the participant subjects reported discomfort upon removing the adhesive compositions using a self-assessed ascending scale from 1 to 5 (1 being the least painful and 5 being the most painful).

The peel force measurements. indicate that the Example D provided appreciably higher adhesive strength (at least 80% greater) at all times after application to the skin. Despite achieving such tenacious adhesive values, the adhesive composition of Example D was surprisingly removed without causing more damage to the skin than the comparative SOFTFLEX® adhesive at all times, as demonstrated by the TEWL data presented in Table II. A smaller increase relative to the comparative SOFTFLEX® adhesive indicates that less damage was caused to the skin surface as a result of removing the test adhesive.

Finally, participants in the study generally perceived less discomfort on removal of the test adhesive, consistent with less damage to the skin surface. These data also support the assertion that the pressure-sensitive adhesive compositions according to the disclosure are more skinfriendly than conventionally available pressure-sensitive adhesive compositions containing naturally-derived hydrocolloids.

TABLE II

| Removal Time | Peel Force from Skin | | TEWL Increase | | Removal Discomfort | |
|---|---|---|---|---|---|---|
| (after application) | grams | | g/m² hour | | Self-assessed 1-5 | |
| Hours | Control | Example D | Control | Example D | Control | Example D |
| 0.5 | 370 | 798 | 0.7 | 0.4 | 1.5 | 1.7 |
| 6 | 317 | 913 | 3.8 | 3.3 | 2.7 | 1.2 |
| 24 | 262 | 693 | 4.7 | 3.3 | 2.8 | 0.8 |
| 48 | 219 | 527 | 2.5 | 1.7 | 2.0 | 1.7 |
| 72 | 113 | 206 | 2.8 | 2.2 | 2.7 | 2.0 |

The foregoing description is given for clearness of understanding only and does not describe every possible embodiment of pressure-sensitive adhesive compositions in accordance with the disclosure; accordingly, no unnecessary limitations should be understood therefrom as modifications may be readily apparent ; to those having ordinary skill in the art.

What is claimed is:

1. A pressure-sensitive adhesive composition comprising:
about 0.25 weight percent (wt. %) to about 15 wt. % of a high molecular weight rubber triblock copolymer;
about 35 wt. % to about 65 wt. % of a superabsorbent material;
0 wt. % to about 15 wt. % of a high molecular weight diblock rubber;
0 wt. % to about 20 wt. % of an end block resin;
0 wt. % to about 40 wt. % of a diluent;
0 wt. % to about 35 wt. % of a solid tackifier;
0 wt. % to about 20 wt. % of a hydrocolloid comprising a natural product or a modified natural product; and,
0 wt. % to about 10 wt. % of a semi-solid hydrocarbon.

2. The pressure-sensitive adhesive composition according to claim 1, wherein the pressure-sensitive adhesive comprises about 35 wt. % to about 60 wt. % of a superabsorbent material.

3. The pressure-sensitive adhesive composition according to claim 1, wherein the pressure-sensitive adhesive composition has gel-like properties.

4. The pressure-sensitive adhesive composition according to claim 3, wherein the pressure-sensitive adhesive composition has a compressive force of less than about 15 pounds per square inch (psi) at 10 percent (%) strain.

5. The pressure-sensitive adhesive composition according to claim 1, wherein the superabsorbent material comprises a polymer.

6. The pressure-sensitive adhesive composition according to claim 1, wherein the pressure-sensitive adhesive composition comprises 0 wt. % to about 10 wt. % of the hydrocolloid comprising a natural product or a modified natural product.

7. The pressure-sensitive adhesive composition according to claim 1, wherein the pressure-sensitive adhesive composition is substantially free of hydrocolloids comprising a natural product or a modified natural product.

8. The pressure-sensitive adhesive composition according to claim 1, further comprising an emollient.

9. An article comprising a layer of the pressure-sensitive adhesive composition according to claim 1.

10. The article according to claim 9, wherein the article is selected from the group consisting of waste collection appliances, dressings, prosthetic devices, metal devices, and plastic devices.

11. A waste collection appliance comprising:
a mounting faceplate; and,
a waste collection pouch comprising a polymeric material,
wherein the mounting faceplate includes a layer of pressure-sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

12. The waste collection appliance according to claim 11, wherein the pressure-sensitive adhesive composition has gel-like properties.

13. The waste collection appliance according to claim 11, wherein the superabsorbent material comprises a polymer.

14. The waste collection appliance according to claim 11, wherein the pressure-sensitive adhesive composition is substantially free of hydrocolloids comprising a natural product or a modified natural product.

15. The waste collection appliance according to claim 11, wherein the pressure-sensitive adhesive composition further comprises an emollient.

16. A pressure-sensitive adhesive composition comprising:
about 0.25 weight percent (wt. %) to about 15 wt. % of a high molecular weight rubber triblock copolymer; and,
about 35 wt. % to about 65 wt. % of a superabsorbent material,
wherein the adhesive composition has a compressive force of less than about 15 pounds per square inch (psi) at 10 percent (%) strain.

17. The pressure-sensitive adhesive composition according to claim 16, wherein adhesive composition has a compressive force of less than about 10.0 psi at 10% strain.

18. An article comprising a layer of the pressure-sensitive adhesive composition according to claim 16.

19. A self-adhering wound dressing comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a liquid diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

20. The self-adhering wound dressing according to claim 19, wherein the wound dressing does not contain a separate component for absorption.

21. The self-adhering wound dressing according to claim 19, wherein the wound dressing is substantially free of hydrocolloids comprising a natural product or a modified natural product.
